(19) United States
(12) Patent Application Publication
(10) Pub. No.: US 2024/0336557 A1
(43) Pub. Date: Oct. 10, 2024

(54) METHOD FOR CONTINUOUSLY RECOVERING DIMETHYLFORMAMIDE FROM WASTE SOLUTION RESULTING FROM PRODUCTION OF POLYIMIDE

(71) Applicant: GTRNR CO., LTD., Incheon (KR)

(72) Inventors: Sun Sub HWANG, Incheon (KR); Seok Jung KIM, Seoul (KR); Baek Gyu HWANG, Incheon (KR); Yong Hyun KIM, Incheon (KR)

(21) Appl. No.: 18/294,789

(22) PCT Filed: Aug. 2, 2022
(86) PCT No.: PCT/KR2022/011402

§ 371 Date: Feb. 2, 2024

(30) Foreign Application Priority Data
Aug. 6, 2021 (KR) 10-2021-0104158

(51) Int. Cl. C07C 231/24; B01D 1/06; B01D 3/14; B01D 3/28; B01D 3/34

(52) U.S. Cl. CPC C07C 231/24; B01D 1/065; B01D 3/148; B01D 3/28; B01D 3/34

(57) ABSTRACT

A method for recovering dimethylformamide from a polyimide production waste solution containing dimethylformamide, acetic acid, a catalyst, and water, includes (a) a neutralization process of forming the acetic acid into a non-volatile acetate by adding a metal hydroxide to the waste solution, (b) a first distillation process of removing the water contained in the waste solution that has gone through the step (a), (c) a second distillation process of separating and removing the non-volatile acetate from the waste solution, (d) a water content control process of controlling the water content in the waste solution by adding water to the waste solution such that the dimethylformamide and the catalyst contained in the distilled waste solution in the step (c) do not form an azeotropic mixture, and (e) a third distillation process of separating and recovering dimethylformamide through fractional distillation from the waste solution in which the water content has been controlled.

### TECHNICAL FIELD

The present invention relates to a method for recovering dimethylformamide at a high purity through a continuous process, which is contained in a waste solution resulting from the production of polyimide.

### BACKGROUND ART

In the field of display devices and the like, such as displays and liquid crystal displays using organic electroluminescent elements, there is a demand for materials having high light transmittance and excellent heat resistance, and at the same time being lightweight and flexible.

Polyimide, developed in response to such a demand, is a material with the most excellent thermal, mechanical, and chemical properties among polymers known so far, and was developed for space and aviation, but is currently widely used in semiconductor and display fields.

For the production of a polyimide film formed by filming polyimide, a method is widely used wherein aromatic dianhydride and aromatic diamine or aromatic diisocyanate are subjected to solution polymerization to prepare a polyamic acid derivative, and then the prepared polyamic acid derivative is cast into a film form, dried, and imidized by ring-closure dehydration at a high temperature.

In the above process, a dianhydride component and a diamine component are synthesized by condensation polymerization in an organic solvent such as dimethylformamide (DMF), and after the synthesis, acetic acid, a reaction catalyst, water, solid particles and the like are contained together with dimethylformamide in a waste solution.

Since a waste solution resulting from the production of a polyimide film contains a large amount of dimethylformamide, a special chemical substance, the waste solution is required to be disposed through a strict treatment procedure in accordance with a law on waste disposal. Currently, a resulting waste solution is treated by incineration, but such a treatment method requires considerable cost, which increases the production cost of a polyimide film, and causes environmental pollution at the same time.

Meanwhile, fractional distillation may be considered as a method for recovering dimethylformamide contained in a waste solution after synthesis, but since a catalyst and acetic acid contained in the waste solution form an azeotropic mixture with water, the dimethylformamide (DMF), etc., it is impossible to separate and recover the dimethylformamide at a high purity high enough to allow the dimethylformamide to be reintroduced into a production process through a simple fractional distillation method.

In this regard, Korean Patent Laid-open Publication No. 2019-0064300 proposes a method for recovering dimethylformamide through a system including a reactive waste solvent filtration device that recovers a reactive waste solvent composed of a mixture of organic, aqueous, and solid particles resulting from a polyimide synthesis process and filters the solid particles contained therein, a preheating device that performs preheating before supplying the reactive waste solvent; a reactive distillation device that adds an alcohol in a gaseous state to the filtered reaction waste solvent to esterify acetic acid contained in the reaction waste solvent, an adsorption device that removes a small amount of unreacted acetic acid and impurities remaining after a neutralization process by allowing the same to pass through an adsorption column, and a fractional distillation device that performs fractional distillation on a material treated with the adsorption column.

However, the above method has a limitation in that it is difficult to economically recover dimethylformamide at a purity high enough to compete with that of dimethylformamide that is not recycled (a purity currently required in the electronics industry is 99.5% or greater).

### DISCLOSURE OF THE INVENTION

### Technical Problem

The purpose of the present invention is to provide a method for efficiently recovering high-purity dimethylformamide through a continuous process from a polyimide production waste solution containing dimethylformamide, acetic acid, a catalyst, and water.

### Technical Solution

In order to achieve the above purpose, the present invention provides a method for recovering dimethylformamide from a polyimide production waste solution containing dimethylformamide, acetic acid, a catalyst, and water, wherein the method includes (a) a neutralization process of forming the acetic acid into a non-volatile acetate by adding a metal hydroxide to the waste solution, (b) a first distillation process of removing the water contained in the waste solution that has gone through the step (a), (c) a second distillation process of separating and removing the non-volatile acetate from the waste solution from which at least a portion of the water has been removed through the step (b), (d) a water content control process of controlling the water content in the waste solution by adding water to the waste solution to avoid the dimethylformamide and the catalyst contained in the distilled waste solution in the step (c) becoming an azeotropic mixture state, and (e) a third distillation process of separating and recovering dimethylformamide from the waste solution by performing fractional distillation on the waste solution in the step (d).

### Advantageous Effects

According to the present invention, dimethylformamide having a purity similar to or equivalent to that of non-recycled dimethylformamide may be recovered through a continuous process from a polyimide production waste solution.

In addition, since the high purity of dimethylformamide recovered according to the present invention is not degraded even after repeated recycling, it is possible to continuously recycle dimethylformamide, an expensive organic solvent, thereby significantly reducing the production cost of polyimide.

In addition, according to an embodiment of the present invention, acetic acid contained in a waste solution is first formed into a non-volatile acetate through a neutralization process, and then a predetermined amount (a portion or all) of water is separated from the waste solution through a film evaporation device, and the acetate is separated and removed from the waste solution using a thin film evaporation device. As described above, by removing the water before removing the acetate from the thin film evaporation device through the film evaporation device, it is possible to reduce a subsequent processing capacity requirement of the thin film device, which enables continuous removal of the acetate at low cost while being efficient throughout a system. Next, dimethylformamide contained in the waste solution is recovered by fractional distillation after controlling the water content in the waste solution by adding water to the waste solution from which the acetate has been removed such that the azeotropic state between the catalyst and the dimethylformamide is dissolved. The above process proceeds continuously, and through the continuous process, the present invention may economically recover dimethylformamide of a high purity of 99% or greater (preferably 99.9% or greater).

In addition, according to the present invention, it is possible to significantly reduce disposal cost of the waste solution, and prevent environmental pollution.

### BRIEF DESCRIPTION OF THE DRAWINGS

FIG. 1 is a diagram of a continuous recovery process of dimethylformamide contained in a polyimide production waste solution according to an embodiment of the present invention.

FIG. 2 shows a dimethylformamide continuous recovery system for performing a process according to an embodiment of the present invention.

### BEST MODE FOR CARRYING OUT THE INVENTION

Hereinafter, preferred embodiments of the present invention will be described in detail with reference to the accompanying drawings. However, the embodiments of the present invention illustrated below may be modified into other various forms, and the scope of the present invention is not limited to the embodiments described below. The embodiments of the present invention are provided to more fully describe the present invention to those skilled in the art.

A method for recovering dimethylformamide from a polyimide production waste solution containing dimethylformamide, acetic acid, a catalyst, and water, the method according to the present invention, is characterized by including (a) a neutralization process of forming the acetic acid into a non-volatile acetate by adding a metal hydroxide to the waste solution, (b) a first distillation process of removing the water contained in the waste solution that has gone through the step (a), (c) a second distillation process of separating and removing the non-volatile acetate from the waste solution from which at least a portion of the water has been removed through the step (b), (d) a water content control process of controlling the water content in the waste solution by adding water to the waste solution to avoid the dimethylformamide and the catalyst contained in the distilled waste solution in the step (c) becoming an azeotropic mixture state, and (e) a third distillation process of separating and recovering dimethylformamide from the waste solution by performing fractional distillation on the waste solution in the step (d).

The method according to the present invention relates to the recovery of dimethylformamide (DMF) through a continuous process with excellent efficiency from a waste solution in which a catalyst and acetic acid form an azeotropic mixture with water, dimethylformamide (DMF), etc., making it difficult to separate the same through fractional distillation.

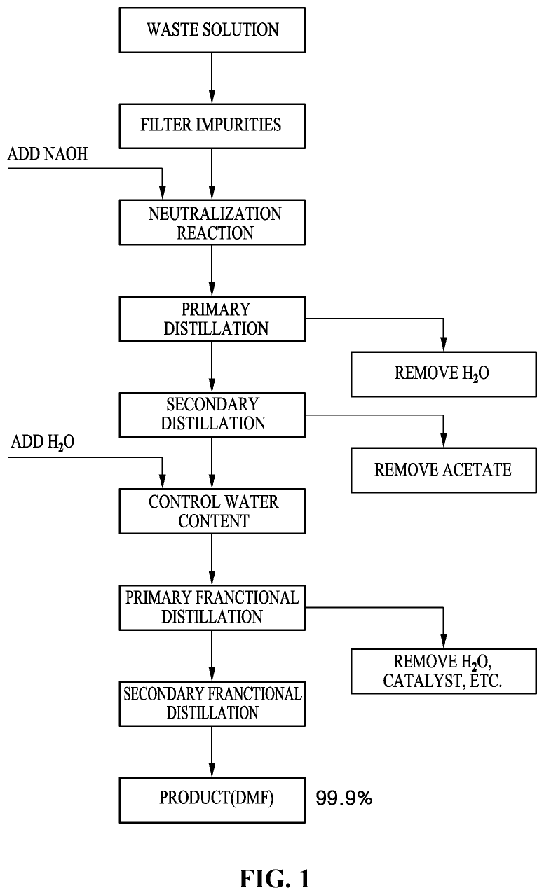

FIG. 1 is a diagram of a continuous recovery process of dimethylformamide contained in a polyimide production waste solution according to an embodiment of the present invention. Referring to FIG. 1, each process will be described in detail.

### 1. Filtration Process

The polyimide production waste solution may contain some impurities in the form of solid particles or floating substances from raw materials or a composite of the raw materials used in a polyimide synthesis process. In addition, dimethylformamide used to dissolve raw materials in the polyimide synthesis process, a catalyst used to promote a synthesis reaction, acetic acid, water, and the like are contained therein.

An acid catalyst or a base catalyst may be used as a catalyst used for the production of polyimide, and for example, the acid catalyst may be p-hydroxyphenylacetic acid, and the base catalyst may be triethylamine, a pyridine-based compound, 1,4-diazabicyclo[2,2,2]octane, 1,8-diazabicyclo[5.4.0]undec-7-ene, etc., but various catalysts may be used without being necessarily limited thereto. The method according to the present invention may be preferably used when a pyridine-based compound is included as a catalyst in the waste solution.

The waste solution may be in a state of containing 50 to 75 wt % of the dimethylformamide, 8 to 20 wt % of the acetic acid, 2 to 10 wt % of the catalyst, and 3 to 20 wt % of the water.

The filtration process is a process of removing various fine floating substances or solid particles contained in the waste solution through a filter. At this time, it is only necessary to use a filter (e.g., a micro filter) that is capable of sufficiently removing fine floating substances, and the filter is particularly limited as long as it can remove the same to a level that does not affect a subsequent process.

The process of FIG. 1 according to an embodiment of the present invention includes the filtration process, but the filtration process may not be performed in cases where the state of a collected waste solution is sufficiently clean without filtering impurities.

### 2. Neutralization Process

The neutralization process is a process of adding a metal hydroxide to the waste solution to change acetic acid contained in the waste solution into a non-volatile acetate through Reaction Equation 1 below.

CH₃COOH+M(OH)n→(CH₃COO)nM+H₂O [Reaction Equation 1]

(Where, M is one selected from the group consisting of Na, K, and Ca, and n is 1 or 2)

It is preferable that the metal hydroxide added for the above reaction has an amount of 95 to 105% of the equivalent of the acetic acid contained in the waste solution, and the waste solution has a pH of 8.5 to 10.5, and it is more preferable that the metal hydroxide added for the reaction has an amount 98 to 100% of the equivalent of the acetic acid, and the pH of the waste solution is 8.8 to 9.5.

Particularly, the pH of the waste solution that has gone through the neutralization process affects both the purity and yield of dimethylformamide recovered through a subsequent distillation process, and if the pH is high, decomposition occurs during the distillation process, which not only significantly lowers the yield but also lowers the purity. In addition, if the pH is low, the purity is significantly degraded.

Accordingly, in order to implement a purity of 99.5% or greater of dimethylformamide currently required by the electronics industry, it is more preferable to maintain the pH of the waste solution after the neutralization process in the range of 8.8 to 9.5.

In addition, sodium hydroxide (NaOH) may be most preferable as the metal hydroxide.

### 3. First Distillation Process

The first distillation process is a process of separating and removing water contained in the waste solution from the waste solution before proceeding with the second distillation process. The process is a process for increasing the efficiency of the entire process by reducing the volume of the waste solution to be treated in the second distillation process, thereby reducing the capacity of equipment required for the second distillation process, and increasing the throughput of the second distillation process.

The first distillation process may be preferably performed using a film evaporation device, but is not necessarily limited thereto. Meanwhile, removing as much water contained in the waste solution as possible through the first distillation process may increase the efficiency of the subsequent second distillation process, but some water may remain even after the first distillation process.

### 4. Second Distillation Process

The second distillation process may be preferably performed using a thin film evaporation device. As a result, it is possible to continuously remove the acetate.

### 5. Water Control Process

The water control process is a process to avoid dimethylformamide and the catalyst becoming an azeotropic mixture state in a mixture of the dimethylformamide, the catalyst, and the water contained in the waste solution from which the acetic acid has been removed through the neutralization process, the first distillation process, and the second distillation process.

This is a step of adjusting the water content in the waste solution to be within 25 to 40 wt % by additionally adding water to the waste solution that has passed through the first distillation process and the second distillation process. If the content of water contained in the waste solution is out of the above range, the azeotropic mixture state formed between the catalyst and the dimethylformamide is not dissolved, so that it is not possible to separate the dimethylformamide at a high purity through a subsequent fractional distillation method.

For example, when dimethylformamide (b.p. 154° C.), 3-methylpyridine (b.p. 144° C.) and water (b.p. 100° C.) are mixed, if the water is contained within the above range, the water and the 3-methylpyridine form an azeotropic mixture, and an azeotropic mixture state of the 3-methylpyridine and the dimethylformamide is broken, so that it is possible to separate only the dimethylformamide through fractional distillation.

Considering the efficiency of the process (reducing the number of times of fractional distillation) and the purity of the recovered dimethylformamide, it is more preferable that the water content in the waste solution is adjusted within the range of 27 to 33 wt % through the water control process.

### 6. Third Distillation Process

The third distillation process is a process of performing fractional distillation to separate the dimethylformamide component contained in the waste solution using the difference in vapor pressure among the components contained in the waste solution in which the water content has been controlled. Since the azeotropic mixture state between the catalyst and the dimethylformamide in the waste solution is broken through the above-described water control process, it is possible to separate the dimethylformamide from the waste solution at a high purity through a fractional distillation process.

The process is performed in a fractional distillation column equipped with a preheater, a fractionation column, a reboiler, a condenser, a receiver, and a reflux ratio control device.

When the waste solution introduced into the fractional distillation column is heated, the catalyst and the water having a relatively low boiling point are removed from the top of the fractional distillation column, and the dimethylformamide is recovered from the bottom of the fractional distillation column.

At this time, since the condensate obtained through the condenser may contain a significant amount of dimethylformamide, by refluxing some of the condensate to the top of the fractionation column, it is possible to reduce the loss of dimethylformamide mixed in the top of the fractionation column. At this time, the reflux ratio of the condensate may be 0.2 to 4.0, preferably 1.0 to 2.0.

Dimethylformamide having a concentration of 99% or greater may be obtained through the third distillation process as described above.

### 7. Fourth Distillation Process

The fourth distillation process is a process selectively performed when it is necessary to further increase the purity of dimethylformamide (DMF) recovered through the third distillation process (e.g., when dimethylformamide obtained through the second distillation process contains impurities of a high boiling point), and may be performed more than once using a fractional distillation column having substantially the same structure as the one in the third distillation process.

At this time, when the dimethylformamide (DMF) added to the fractional distillation column is heated, the dimethylformamide is recovered from the top of the fractional distillation column, and a small amount of the impurities of a high boiling point is removed from the bottom of the fractional distillation column.

In addition, the fourth distillation process may be performed under atmospheric pressure or under vacuum, and may preferably be performed under a vacuum of 50 to 300 torr to suppress the decomposition of dimethylformamide (DMF) at a high temperature.

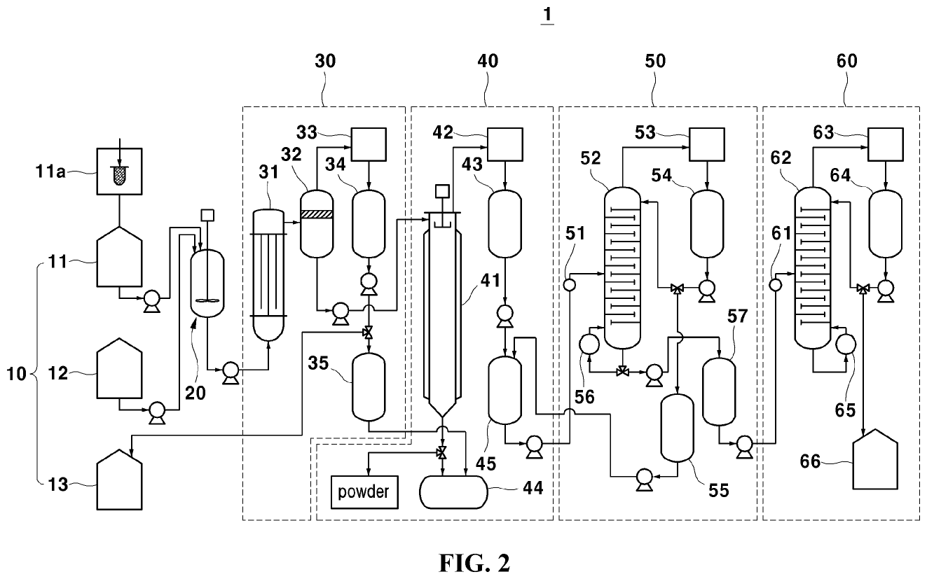

FIG. 2 shows a dimethylformamide recovery system for performing a process according to an embodiment of the present invention. Hereinafter, a dimethylformamide recovery system 1 for performing the above-described process will be described in detail with reference to FIG. 2.

The dimethylformamide recovery system 1 according to an embodiment of the present invention largely includes a storage tank 10, a neutralization device 20, a first distillation device 30, a second distillation device 40, a primary fractional distillation device 50, and a secondary fractional distillation device 60.

The storage tank 10 includes a waste solution storage tank 11 for storing a waste solution, a neutralizer storage tank 12, and a wastewater/waste solution storage tank 13 for storing wastewater/waste liquid generated in the recovery system. Among them, a filter device 11a for removing solid particles or various fine floating substances contained in the waste solution is provided on the side of an inlet of the waste solution storage tank 11. In addition, an outlet of the waste solution storage tank 11 and an outlet of the neutralizer storage tank 12 are connected to an inlet of the neutralization device 20, so that the waste solution or a neutralizer may be transferred to the neutralization device 20.

The neutralization device 20 is a device for neutralizing the acetic acid contained in the waste solution, and includes a tank and a stirrer provided in the tank. An outlet of the neutralization device 20 is connected to the first distillation device 30.

The first distillation device 30 includes a film evaporator 31, a water separation tank 32, a water condenser 33, a water condensation tank 34, and a condensate storage tank 35. Among them, the film evaporator 31 is an evaporator in which a plurality of evaporation tubes are installed inside a heating column extending vertically, and is a device in which the water contained in the waste solution is allowed to evaporate when the waste solution is supplied from the bottom, the evaporated water vapor is allowed to rise along the center of the evaporation tube, and the remaining non-evaporated liquid is allowed to rise along the tube wall while forming a thin film. It is possible to separate a significant amount of the water contained in the waste solution through the above device. In addition, the water separation tank 32 is a tank for separating evaporated water and a non-evaporated liquid material, the condenser 33 is a device for condensing water vapor separated through the water separation tank 32, the condensation tank 34 is a tank for receiving water condensed through the condenser 33, and the condensate storage tank 35 is a tank for storing the condensed water.

The second distillation device 40 includes a thin film evaporation device 41, a waste solution condenser 42, a waste solution storage tank 43, a waste solution mixing tank 44, and a water content control tank 45.

The thin film evaporator 41 is a device that heats and evaporates the waste solution while the waste solution flows in the form of a thin film, and at the top of the evaporator, stirring blades rotating at a high speed causes the waste solution to form a thin film on the inner surface of the column. The waste solution condenser 42 is a device for condensing a gas evaporated by the thin film evaporator 41, and the waste solution storage tank 43 is a tank for storing a liquid condensed in the waste solution condenser 42. The waste liquid mixing tank 44 is a tank for mixing a waste liquid, from which powder-phase acetate in a powder phase has been removed, among materials discharged from the bottom of the thin film evaporator 41 and water in the condensate storage tank 35.

The water content control tank 45 is a tank for controlling the content of water by adding water to the waste solution in which the gas evaporated from the thin film evaporator 41 is condensed. In the water content control tank 45, the content of the water contained in the waste solution is controlled to be in the range of 25 to 40 wt %.

The primary fractional distillation device 50 includes a preheater 51, a primary fractional distillation column 52, a primary fractional condenser 53, a primary fractional condensate circulation tank 54, and a primary fractional condensate storage tank 55, a primary fractional distillation column reboiler 56, and a primary fractional distillation product storage tank 57.

Among them, the preheater 51 is a device for preheating a solution to be introduced into the primary fractional distillation column 52. The first fractional distillation column 52 is a device that separates and extracts the dimethylformamide from the water and the catalyst from the waste solution. The catalyst and the water in the waste solution preheated in the preheater 51 and then transferred to the primary fractional distillation column 52 are discharged from the top of the primary fractional distillation column 52 and the dimethylformamide separated by the primary fractional distillation column 52 is discharged from the bottom and stored in the primary fractional product storage tank 57. The primary fractional condenser 53 is for condensing the catalyst and the water discharged from the top, and the primary fractional condensate circulation tank 54 is a circulation tank for refluxing some of the condensate condensed by the primary fractional condenser 53 to the primary fractional distillation column 52. The primary fractional condensate storage tank 55 is a tank for storing the condensate of the catalyst and the water, and some of the condensate stored in the tank may be transferred to the distillation tank 31 and the distillation solution control tank 34 to allow the recovery process to proceed again. The primary fractional distillation column reboiler 56 is a device for reheating the dimethylformamide-concentrated liquid discharged from the bottom of the primary fractional distillation column 42 and introducing the reheated liquid into the fractional distillation column.

The secondary fractional distillation device 60 includes a preheater 61, a secondary fractional distillation column 62, a secondary fractional condenser 63, a secondary fractional condensate circulation fractional condensate storage tank 64, a secondary fractional distillation column reboiler 65, and a secondary fractional distillation product storage tank 66.

Among them, the preheater 61 is a device for preheating a dimethylformamide solution to be introduced into the secondary fractional distillation column 62. The secondary fractional distillation column 62 is a device for separating and purifying trace amounts of impurities contained in the dimethylformamide solution, and separates trace amounts of high-boiling point materials contained in the dimethylformamide. At this time, the dimethylformamide in the dimethylformamide solution transferred to the secondary fractional distillation column 62 is discharged from the top of the secondary fractional distillation column 62, and the impurities and a trace amount of the dimethylformamide are discharged from the bottom. The secondary fractional condenser 63 is for condensing the dimethylformamide discharged from the top, and the secondary fractional condensate circulation tank 64 is a circulation tank for refluxing some of the dimethylformamide condensed by the secondary fractional condenser 63 to the secondary fractional distillation column 62. The secondary fractional distillation column reboiler 65 is a device for reheating a liquid in which impurities and dimethylformamide are concentrated and introducing the reheated liquid into the fractional distillation column. The secondary fractional distillation product storage tank 66 is a tank for storing a refined final product.

### Examples

100 kg of a waste solution resulting from the production of a polyimide film for a flexible display was collected. The analysis of components of the collected waste solution confirmed that 73.0 wt % of dimethylformamide (DMF), 21.0 wt % of acetic acid, 2.1 wt % of 3-methylpyridine, and 3.9 wt % of water were contained.

First, the waste solution was passed through a microfilter to remove solid particles or floating substances contained in the waste solution. Thereafter, the waste solution was introduced into the neutralization device 20 in which a stirrer was installed, and caustic soda (NaOH) having a concentration of 50% was added thereto until the pH of the waste solution reached 9.0. Moisture after the neutralization was analyzed to be 24.4 wt %.

As described above, the waste solution neutralized by the addition of the caustic soda was introduced at a flow rate of 1 liter per minute while being heated with a heat medium at 150 to 160° C. under a vacuum of 100 torr using a film distillation device, which is the first distillation device 30. Under the above condition, it was possible to remove up to 6.5 wt % of moisture in content contained in the waste solution.

Thereafter, the waste solution from which a portion of the moisture has been removed was passed through a thin film distillation device, which is the second distillation device 40, while being heated (heat medium circulation at 150 to 160° C.) under a vacuum (30 to 40 torr) to evaporate and condense dimethylformamide, a catalyst, and residual moisture, and sodium acetate contained in the waste solution was separated and removed in a powder phase.

Thereafter, a total of 27.5 kg of water was added to the waste solution (moisture content of 6.5 wt %), which was subjected to the above second distillation process, such that the content of water was to be 31 to 32 wt % to control the moisture.

The solution with the adjusted water content as described above was subjected to fractional distillation using the primary fractional distillation device 50. Specifically, after preheating was performed to 80 to 85° C. through a preheater, the solution was injected at a rate of 1 liter per minute into the middle of the primary fractional distillation column 52 in which a wire-mesh ring was installed, and a reboiler was maintained at 155 to 158° C. to continuously perform fractional distillation. At this time, the catalyst and the water were evaporated together from the top of the primary fractional distillation column 52 and continuously condensed through the primary fractional condenser 53, and some of the condensate was refluxed to the top of the primary fractional distillation column 52. At this time, the reflux ratio was adjusted to 2:1 to 4:1, and the temperature at the top of the primary fractional distillation column was maintained at 98 to 100° C.

Dimethylformamide (DMF) recovered through the above-described process was subjected to fractional distillation using the secondary fractional distillation device 60. Specifically, after preheating was performed to 80 to 85° C. through a preheater, the dimethylformamide was injected at a rate of 2 liter per minute into the middle of the primary fractional distillation column 62 in which a wire-mesh ring was installed, and a reboiler was maintained at 92 to 95° C. to continuously perform fractional distillation under a vacuum of 95 to 98 torr. At this time, the dimethylformamide was evaporated from the top of the secondary fractional distillation column 62 and continuously condensed through the secondary fractional condenser 63, and some of the condensate was refluxed to the top of the secondary fractional distillation column 62. At this time, the reflux ratio was adjusted to 2:1 to 4:1, and the temperature at the top of the primary fractional distillation column was maintained at 88 to 90° C.

Dimethylformamide (DMF) collected in the second fractional condensate circulation tank 64 at the top of the secondary fractional distillation column 62 had a purity of 99.9%, and a total of 69.1 Kg of the dimethylformamide (94.6% yield) was obtained.

### DESCRIPTION OF THE REFERENCE NUMERALS OR SYMBOLS

1: Dimethylformamide recovery system
10: Storage Tank
20: Neutralization device
30: Film distillation device
40: Thin film distillation device
50: Primary fractional distillation device
60: Secondary fractional distillation device

1. A method for recovering dimethylformamide from a polyimide production waste solution containing dimethylformamide, acetic acid, a catalyst, and water, the method comprising:
(a) a neutralization process of forming the acetic acid into a non-volatile acetate by adding a metal hydroxide to the waste solution;
(b) a first distillation process of removing the water contained in the waste solution that has gone through the step (a);
(c) a second distillation process of separating and removing the non-volatile acetate from the waste solution from which at least a portion of the water has been removed through the step (b);
(d) a water content control process of controlling the water content in the waste solution by adding water to the waste solution to avoid the dimethylformamide and the catalyst contained in the distilled waste solution in the step (c) becoming an azeotropic mixture state; and
(e) a third distillation process of separating and recovering dimethylformamide from the waste solution by performing fractional distillation on the waste solution in the step (d).

2. The method of claim 1, wherein prior to the step (a), a filtration process of filtering and removing solid particles or floating substances contained in the waste solution is performed using a filter.

3. The method of claim 1, wherein in the step (a), the metal hydroxide is a material represented by Equation below:
M(OH)n [Equation 1]
(where, M is one selected from the group consisting of Na, K, and Ca, and n is an integer of 1 or 2)

4. The method of claim 1, wherein in the step (b), the first distillation process is performed through a film evaporation device.

5. The method of claim 1, wherein in the step (c), the second distillation process is performed through a thin film distillation device.

6. The method of claim 1, wherein in the step (d), water is added such that the water content in the waste solution is 25 to 40 wt % of the total weight of the waste solution.

7. The method of claim 1, further comprising a fourth distillation process for purifying dimethylformamide obtained through the third distillation process.

8. The method of claim 2, wherein in the step (a), the metal hydroxide is a material represented by Equation below:
M(OH)n [Equation 1]
(where, M is one selected from the group consisting of Na, K, and Ca, and n is an integer of 1 or 2)

9. The method of claim 2, wherein in the step (b), the first distillation process is performed through a film evaporation device.

10. The method of claim 2, wherein in the step (c), the second distillation process is performed through a thin film distillation device.

11. The method of claim 2, wherein in the step (d), water is added such that the water content in the waste solution is 25 to 40 wt % of the total weight of the waste solution.

12. The method of claim 2, further comprising a fourth distillation process for purifying dimethylformamide obtained through the third distillation process.
